(19) United States
(12) Patent Application Publication
(10) Pub. No.: US 2005/0159796 A1
(43) Pub. Date: Jul. 21, 2005

(54) Head covering with a flexible conformational array of light for stimulating hair growth

(76) Inventor: Avigdor Ronn, Great Neck, NY (US)

Correspondence Address: AVIGDOR RONN, 27A BOND ST, GREAT NECK, NY, 11021, US

(21) Appl. No.: 11/039,499

(22) Filed: Jan. 20, 2005

Related U.S. Application Data
(60) Provisional application No. 60/538,003, filed on Jan. 21, 2004.

(51) Int. Cl. A61N 1/00

(52) U.S. Cl. 607/88

(57) ABSTRACT

An area of hair-loss is treated by subjecting the affected area to a flexible array of light emitting units that conforms to the contour of the affected hair-loss area, thereby delivering a therapeutic dose of light to that affected hair-loss area. A flexible array of light emitting units is conformingly placed onto the hair-loss area. The light emitting units are energized to deliver a therapeutic dose of light over the hair-loss area. Treatment can be repeated as needed.

### BACKGROUND OF THE INVENTION

The present invention relates to the treatment of scalp hair-loss by subjecting the scalp to low intensity light. In particular, the present invention is directed to the treatment of scalp hair-loss by subjecting the affected area to a flexible array of a plurality of light emitting units attached within a head covering such as a hat or cap. The flexible array conforms—with the assistance of the head covering—to the contour of the affected scalp area, thereby delivering a therapeutic dose of light to that scalp area. The present invention is also directed to a hat that includes a detachable flexible array of a plurality of light emitting units with a portable power supply.

Laser biostimulation, also known as low level laser therapy (LILT) is a technique used to alleviate localized pain. Furthermore, the therapy is effective to perform photorejuvenation, wrinkle removal, collagen remodeling, as well as general muscular and holistic relaxation. Without being limited by the theory, it is now believed to stimulate hair growth by increasing blood flow and thereby assist in improving the general health of the skin. The therapy delivers 1-20 J/cm² of energy at low intensity (1-30 mW/cm²) to the area of the body requiring treatment. Presently, a low power laser is directed at an area for a predetermined time. Then, the laser is moved to an adjacent area and the laser is shone onto that area for the predetermined time. This process is repeated until the whole body area at which the pain is localized has been treated by the laser. Although the time that the laser is shone onto the patient for each spot may be only a few minutes, the total time can be uncomfortably long. Or, an array of lasers set in an apparatus similar to a hair-dryer is placed over a subject's head and the subject must sit under the apparatus for a predetermined time.

Thus, it would be desirable to provide a method and apparatus that can treat the scalp of a subject without the need continually to move a single laser from spot to spot during treatment. This would alleviate the discomfort and inconvenience associated with the present dependence on the small spot size of conventional lasers.

Further, it would be desirable to provide an array of a plurality of light emitting units, that can conform to an individual's head shape and size to treat conveniently that individual's scalp area without the need continually to move a single laser from spot to spot during treatment. The plurality of light emitting units can emit at any effective wavelength. Finally, it would be desirable to provide such an array in a portable apparatus that allows free movement of the subject without being tethered or fixed to a chair or treatment facility.

U.S. Pat. No. 6,096,066 describes a conformal patch for administering light therapy to subcutaneous tumors. U.S. Pat. No. 5,997,569 describes a flexible and adjustable grid for medical therapy. U.S. Pat. No. 6,497,719 describes a laser comb. U.S. Pat. No. 2,397,757 describes a hairbrush. U.S. Pat. No. 4,653,495 describes a laser medical apparatus. U.S. Pat. No. 5,103,073 describes a device for laser treatment of an object. U.S. Pat. No. 5,303,722 describes an apparatus for bleaching hair under the influence of light. U.S. Pat. No. 5,569,368 describes electrophoretic apparatus and method for applying therapeutic cosmetic and dyeing solutions to hair. U.S. Pat. No. 5,569,929 describes double-beam light source apparatus, position detecting apparatus and aligning apparatus. U.S. Pat. No. 5,803,093 describes hair/scalp treatment device. U.S. Pat. No. 5,814,078 describes a method and apparatus for regulating and improving the status of development and survival of living organisms. U.S. Pat. No. 6,022,345 describes method and apparatus for laser assisted hair transplantation. U.S. Pat. No. 6,129,748 describes apparatus for applying pulsed light to the forehead of a user. U.S. Pat. No. 6,187,029 describes a photo thermal treatment device.

U.S. Pat. No. 6,666,878 describes method and device stimulating the activity of hair follicles. U.S. Pat. No. 5,616,140 describes method and apparatus for therapeutic laser treatment. U.S. Pat. No. 5,228,431 describes drug-free method for treatment of the scalp for therapeutic purposes. U.S. Pat. No. 6,063,108 describes method and apparatus for localized low energy photon therapy (LEPT). U.S. Pat. No. 6,234,069 describes apparatus in an infuser for a liquid food product. U.S. Pat. No. 6,383,176 describes hair removal device and method.

### SUMMARY OF THE INVENTION

The present invention treats an area of hair-loss by subjecting the affected area to a flexible array of light emitting units that conforms to the contour of the affected hair-loss area, thereby delivering a therapeutic dose of light to that affected hair-loss area. A flexible array of light emitting units is conformingly placed onto the hair-loss area. The light emitting units are energized to deliver a therapeutic dose of light over the hair-loss area. Treatment can be repeated as needed.

The present invention treats scalp hair loss by subjecting the affected scalp to a flexible array comprising a plurality of light emitting units in which the array is attached at least partially within a head covering such as a hat, scarf, or cap. The flexible array can conveniently comprise a multitude of solid state lasers that substantially conform to the contour of the affected scalp, thereby delivering a therapeutic hair-stimulating dose of laser light to that scalp area. The flexible array is assisted in conforming to the scalp area by the action of the hat or cap pressing the array onto the head. Thus, a flexible array of lasers is conformingly placed onto the scalp area. The lasers are energized by a portable or fixed power unit to deliver a therapeutic dose of laser light over the scalp area. The lasers can emit at any convenient therapeutically effective wavelength.

The flexible array can comprise a plurality of light emitting diodes (“LED”) that substantially conforms, with the assistance of the head covering such as a hat, scarf, or cap, to the contour of the affected scalp area to deliver a hair-stimulating dose of electromagnetic energy in the form of light to that affected scalp area. The LEDs are energized by a portable or fixed power supply to deliver a therapeutic dose of light over the scalp area. The LEDs can emit at any convenient therapeutically effective wavelength.

### DETAILED DESCRIPTION OF THE INVENTION

The present invention treats scalp hair loss by:

1) Providing an array of light emitting diodes (“LED”) mounted in a supporting flexible medium such as, for example, an elastomeric polymer. The array is attached to a head covering and to a portable power supply. In one embodiment, the flexible medium is a flexible circuit board material. In another embodiment, the array is detachably attached to the head covering by a hook and loop fasteners such as VELCRO. In another embodiment, the array is sewn onto the head covering. In another embodiment, the array is pinned onto the inside of the head covering. In another embodiment, the array is riveted onto the head covering. In another embodiment, the array is attached by glue or flexible mastic. In an embodiment, the array is covered with a washable material such as polyurethane.

The outputs of the LEDs are advantageously in the 620 nm to 690 nm wavelength region. The output of the LEDs should overlap spatially to assure treatment of the entire affected scalp area, although it is not critical that the entire scalp affected area be treated at once. As much as 40% of the affected scalp area can be without light, although it is advantageous to cover at least 70% of the affected area. Even more advantageous, the LED light output should overlap each other to cover at least 85% of the affected area. The electrical requirements of the LEDs can be supplied by wire leads that connect to a power supply and a controller. The power supply can be battery or line. If to a line power supply, the power leads should be of sufficient length to allow movement of the user about the room. Advantageously, the power supply should be a battery supply. The battery supply can be rechargeable or disposable. The power output of the LEDs should be controlled to yield about 1-3 mW/cm². Such LED units are commercially available from, for example, Agilent Technologies, Inc., Phillips Electronics, SDL Inc, and others. The affected scalp area should be subjected to the LED output delivered until a predetermined energy total has been reached. For example, at an intensity of about 10 mW/cm² for about 8 minutes. The energy can be, for example, 5 J/cm². The LEDs can be incorporated into the devices described in U.S. Pat. No. 6,096,066 or U.S. Pat. No. 5,997,569 and attached at least partially to the inside of a head covering, such as a cap, scarf, helmet, or hat. In one embodiment the array is attached within a helmet to allow hair loss treatment while riding a bicycle, motorcycle, or in-line skate.

2) Bringing the above array of LEDs into conforming proximity to the affected scalp area with hair loss by putting the head covering onto the subject's head;

3) Maintaining the array of LEDs in a conforming proximity geometry to the affected scalp area by keeping the head covering on for a predetermined time period; and

4) Subjecting the affected scalp area to about 1-20 J/cm² of energy by turning the LEDs on for a predetermined time period. As an example, the energy can be delivered at an intensity of about 10 mW/cm² for about 8 minutes.

As described above, the affected scalp area should be subjected to about 1-20 J/cm² of energy. 5 J/cm² of energy can be delivered, for example, at an intensity of about 10 mW/cm² for about 8 minutes. The energy can be in the range of about 1 J/cm² to about 8 J/cm².

The intensity can be in the range of about 1 mW/cm² to about 25 mW/cm². Such intensities are well below the intensity threshold that causes pain. For most people, that intensity threshold is about 150 mW/cm². Accordingly, the intensity should be below 150 mW/cm². The area should be subjected to the LED array's output for about 5 to about 40 minutes. For example, the area may be subjected to the light output for about 8 minutes. Under the parameters described, the light energy will not significantly heat normal scalp and hair. Thus, the hair stimulation treatment will not adversely affect normal scalp temperature.

The present invention also is directed to a head covering such as a hat, scarf, helmet, or cap containing an array of LEDs mounted in a supporting flexible medium. The array is attached at least partially within the head covering. The supporting flexible medium can be, for example, an elastomeric polymer sheet, a network of flexible ribbons, a cloth quilting, a woven mat, or an interlocking network of solid subunits similar to armor chain mail. The outputs of the LEDs are advantageously in the 620 nm to 690 nm wavelength region. The array should distribute the LEDs so that the output of the LEDs overlap spatially to assure treatment of the entire affected scalp area, although it is not critical that the entire scalp affected area be treated at once. As much as 40% of the affected scalp area can be without light, although it is advantageous to cover at least 70% of the affected area. Even more advantageous, the LED light output should overlap each other to cover at least 85% of the affected area. The electrical requirements of the LEDs can be supplied by wire leads that connect to a power supply and a controller. The power supply can be battery or line. When attached to a line source (including a battery charger), the wire leads should be of sufficient length to allow the user to move about the room. Advantageously, the power supply should be a battery supply. The battery supply can be rechargeable or disposable. The power output of the LEDs should be controlled to yield about 1-30 mW/cm². Such LED units are commercially available from, for example, Agilent Technologies, Inc. The affected scalp area should be subjected to the LED output delivered until a predetermined energy total has been reached. For example, at an intensity of about 10 mW/cm² for about 8 minutes. The LEDs can be incorporated into the devices described in U.S. Pat. No. 6,096,066 or U.S. Pat. No. 5,997,569 and attached at least partially to the inside of a head covering such as a cap, scarf, or hat. In one embodiment the array is attached within a helmet to allow hair loss treatment while riding a bicycle, motorcycle, or in-line skate.

The treatment method can be repeated as needed. For example, the treatment is conveniently taken every day for 1/2 hour while exercising.

The present invention also treats scalp hair loss by:

1) Providing an array of solid state lasers mounted in a supporting flexible medium such as, for example, an elastomeric polymer attached to the inside of a hat or cap. The outputs of the lasers are advantageously in the red to near infrared wavelengths. It is convenient to use laser light at 630 nm, 635 nm, 652 nm, 665 nm, 670 nm, 690 nm, 740 nm, 830 nm, or 980 nm. The output of the lasers should overlap spatially to assure treatment of the entire affected scalp hair loss area, although it is not critical that the entire affected scalp be treated at once. As much as 40% of the affected scalp area can be without light, although it is advantageous to cover at least 70% of the affected area. Even more advantageous, the laser light output should overlap each other to cover at least 85% of the affected area. The electrical requirements of the lasers can be supplied by wire leads that connect to a power supply and a controller. The power supply can be battery or line. When attached to a line source, the wire leads should be of sufficient length to allow movement of the user about the room. Advantageously, the power supply is a battery supply for portability. The power output of the lasers should be controlled to yield about 1-150 mW/cm². It is advantageous that the solid-state lasers of the array are sized to subject the scalp area to about 2-8 J/cm² of energy (such as, for example, 4.8 J/cm²) delivered at an intensity of about, for example, 10 mW/cm² for about 8 minutes. Such lasers can be obtained from, for example, Boston Laser, Hitachi, Lumenix, or Panasonic Corp. The lasers can be incorporated into the devices described in U.S. Pat. No. 6,096,066 or U.S. Pat. No. 5,997,569 and attached to the inside of a head covering such as a hat, cap, scarf, or helmet.

2) Bringing the above array of solid-state lasers into conforming proximity to the affected scalp area by putting the head covering onto the head of the subject;

3) Maintaining the array of solid-state lasers in a conforming proximity geometry to the affected scalp area by keeping the hat, cap, or helmet on for a predetermined length of time; and

4) Subjecting the scalp hair loss area to about 2-8 J/cm² of laser energy. As an example, the energy can be delivered at an intensity of about 10 mW/cm² for about 8 minutes.

The array of solid-state lasers is maintained in a conforming proximity geometry to the scalp hair loss area by the urging action of the head covering such as a cap, hat or helmet. As described above, the hair loss area may be subjected to about 4.8 J/cm² of energy delivered at, for example, an intensity of about 10 mW/cm² for about 8 minutes. The energy can be in the range of about 2 J/cm² to about 8 J/cm². Advantageously, the energy should be between about 3.5 J/cm² to about 6.5 J/cm².

Conveniently, the intensity should be from about 1 mW/cm² to about 150 mW/cm². Advantageously, the intensity can be about 1 mW/cm² to about 25 mW/cm². The area should be subjected to the laser array's laser output for about 8 minutes. Advantageously, the area should be subjected to the light output for about 5 to about 10 minutes.

Further, it is convenient to use laser light at the wavelength between about 600 nm and about 1000 nm. It is particularly convenient to use laser light at the wavelength of 630 nm, 635 nm, 652 nm, 665 nm, 670 nm, 690 nm, 740 nm, 830 nm, or 980 nm.

The present invention also is directed to an array of solid state lasers mounted in a supporting flexible medium. The supporting flexible medium can be, for example, an elastomeric polymer sheet, a network of flexible ribbons, a cloth quilting, a woven mat, or an interlocking network of solid subunits similar to armor chain mail. It is advantageous to use laser light at 630 nm, 635 nm, 652 nm, 665 nm, 670 nm, 690 nm, 740 nm, 830 nm, or 980 nm. The output of the lasers should overlap spatially to assure treatment of the entire affected scalp area, although it is not critical that the entire affected scalp be treated at once. The electrical requirements of the lasers can be supplied by wire leads that connect to a power supply and a controller. The power supply advantageously is a battery. The power output of the lasers should be controlled to yield about 1-30 W/cm². It is advantageous that the solid-state lasers of the array are sized to subject the scalp area to about 9.6 J/cm² of energy delivered at an intensity of about 20 mW/cm² for about 8 minutes.

The treatment method can be repeated as needed. For example, the treatment is taken conveniently twice a day for thirty days.

It is apparent that certain wavelengths may be more efficacious than others. The present invention includes the use of any efficacious wavelength as would be known or revealed by experiments commonly performed in the art.

Thus, in one aspect the present invention is directed to a device for treating scalp hair-loss comprising a head covering, a flexible array of a multiplicity of light sources, with the flexible array attached to the head covering, wherein the head covering acts to urge at least a portion of the array into a proximate alignment to a scalp treatment area.

In an embodiment of the aspect, the device further comprises a power source to supply energy to the light sources.

In another embodiment of the aspect, the device further comprises wire leads connecting a power source to at least one of the light sources.

In still another embodiment of the aspect, the head covering is a scarf.

In yet still another embodiment of the aspect, the head covering is a cap or hat.

In another embodiment of the aspect, the head covering is a helmet.

In yet another embodiment of the aspect, the flexible array is detachably attached to the head covering.

In another embodiment of the aspect, the flexible array is attached to the head covering by a hook and loop fastening.

In still another embodiment of the aspect, the flexible array is attached to the head covering by a staple, rivet, or thread.

In yet another embodiment of the aspect, the flexible array is attached to the head covering by a mastic, glue, or cement substance.

In yet still another embodiment of the aspect, the light sources are light emitting diodes.

In another embodiment of the aspect, the light sources are light emitting diodes having output peak wavelengths from about 620 to about 660 nm.

In another embodiment of the aspect, the light sources are lasers having output peak wavelengths at 630 nm, 635 nm, 652 nm, 665 nm, or 670 nm.

In another embodiment of the aspect, the power source is a battery.

In a second aspect, the present invention is directed to a method of treating an area of hair-loss on a mammal, such as a human, comprising the steps of:
providing an array of light emitting diodes (LEDs) mounted in a supporting flexible medium;
bringing said array of LEDs into conforming proximity to the area of hair-loss;
maintaining said array of LEDs in a conforming proximity geometry to the area of hair-loss; and
subjecting the area of hair-loss to about 1-20 J/cm² of energy by energizing the LEDs.

In a third aspect, the present invention is directed to a method of treating an area of hair-loss on a mammal, such as a human, comprising the steps of:
providing an array of solid state lasers mounted in a supporting flexible medium;
bringing said array of solid-state lasers into conforming proximity to the area of hair-loss;
maintaining said array of solid-state lasers in a conforming proximity geometry to the area of hair-loss; and
subjecting the area of hair-loss to about 2-8 J/cm² of energy from the lasers.

In an EXAMPLE 1, 16 LEDs are distributed in an array of 4 rows of 4 on an approximately 1.75 inch by 1.75 inch flexible polymeric circuit board. Each LED had a peak wavelength of about 642 nm. The array is attached inside a cap at a location corresponding to a bald or thinning spot on the subject's scalp. The array is powered by a variable voltage source that allows for tuning of the power output of the LEDs. The variable voltage source is plugged into a line voltage source. The cap and array is sufficiently light and the electric cord is sufficiently long that the subject can conduct normal activities in the room while the scalp treatment is ongoing.

In an EXAMPLE 2, 32 LEDs are distributed evenly in 4 rows of 8 on an approximately 1.75 inch by 4 inch flexible polymeric circuit board. Each LED had a peak wavelength of about 642 nm. The single array had an irradiance of about 1-3 mW/cm2 powered by a 9v battery. The array is sewn onto the inside of a cap at a location corresponding to a bald spot of the scalp. Wire leads are sewn leading to a 9v battery holder on the cap. The cap is worn so the array is proximate to the bald spot and the power is turned on with a switch in the circuit.

In EXAMPLE 3, three of the single panels from EXAMPLE 2 (totaling 96 LEDs) are fastened next to each other and attached to a cap by hook and loop fastening sheets glued to the backs of the panels. Wire leads are pinned to the cap and attached to a battery holder worn on the belt of the subject. The cap is worn so that the three panels are proximate to an area of hair loss on the scalp and the unit is turned on with a switch in the 9v battery pack, which may be rechargeable.

1. A device for treating scalp hair-loss comprising:
a head covering;
a flexible array of a multiplicity of light sources; and
said flexible array attached to said head covering, wherein said head covering acts to urge at least a portion of said array into a proximate alignment to a scalp treatment area.

2. The device according to claim 1, further comprising:
a power source to supply energy to said light sources.

3. The device according to claim 2, further comprising:
wire leads connecting said power source to at least one of said light sources.

4. The device according to claim 1, wherein said head covering is a scarf.

5. The device according to claim 1, wherein said head covering is a cap or hat.

6. The device according to claim 1, wherein said head covering is a helmet.

7. The device according to claim 1, wherein said flexible array is detachably attached to said head covering.

8. The device according to claim 1, wherein said flexible array is attached to said head covering by a hook and loop fastening.

9. The device according to claim 1, wherein said flexible array is attached to said head covering by a staple, rivet, or thread.

10. The device according to claim 1, wherein said flexible array is attached to said head covering by a mastic, glue, or cement substance.

11. The device according to claim 1, wherein said light sources are light emitting diodes.

12. The device according to claim 1, wherein said light sources are light emitting diodes having output peak wavelengths from about 620 to about 690 nm.

13. The device according to claim 1, wherein said light sources are lasers having output peak wavelengths at 630 nm, 635 nm, 652 nm, 665 nm, 670 nm, 740 nm, 830 nm, or 980 nm.

14. The device according to claim 2, wherein said power source is a battery.

15. A method of treating an area of hair-loss on a mammal comprising the steps of:
providing an array of light emitting diodes (LEDs) mounted in a supporting flexible medium;
bringing said array of LEDs into conforming proximity to the area of hair-loss;
maintaining said array of LEDs in a conforming proximity geometry to the area of hair-loss; and
subjecting the area of hair-loss to about 1-20 J/cm² of energy by energizing the LEDs.

16. A method of treating an area of hair-loss on a mammal comprising the steps of:
providing an array of solid state lasers mounted in a supporting flexible medium;
bringing said array of solid-state lasers into conforming proximity to the area of hair-loss;
maintaining said array of solid-state lasers in a conforming proximity geometry to the area of hair-loss; and
subjecting the area of hair-loss to about 2-8 J/cm² of energy from the lasers.
